(19) United States
(12) Patent Application Publication
(10) Pub. No.: US 2013/0189699 A1
(43) Pub. Date: Jul. 25, 2013

(54) IONIC SIGNAL ENHANCEMENT

(71) Applicants: Chung Pei Ou, Singapore (SG); Chan-ju Wang, London (GB); Daniel Morley, London (GB); Sam Reed, London (GB)

(72) Inventors: Chung Pei Ou, Singapore (SG); Chan-ju Wang, London (GB); Daniel Morley, London (GB); Sam Reed, London (GB)

(73) Assignee: DNA Electronics Limited, London (GB)

(21) Appl. No.: 13/878,315

(22) PCT Filed: Oct. 10, 2011
(86) PCT No.: PCT/EP11/67661

§ 371 Date: Apr. 8, 2013

(30) Foreign Application Priority Data
Oct. 8, 2010 (GB) 1017005.8
Oct. 8, 2010 (GB) 1017008.2

(51) Int. Cl. C12Q 1/68

(52) U.S. Cl. CPC C12Q 1/6858; C12Q 1/6869
USPC 435/6.11; 435/6.1

(57) ABSTRACT

Provided is a method of identifying an unknown nucleic acid comprising the steps of combining the unknown polynucleic acid with known nucleic acid reagents in a reaction chamber; producing a first quantity of protons from a polymerisation reaction when bases of one or more of the unknown nucleic acids are complementary to the bases of one or more known nucleic acids comprised within the known reagents; producing a second quantity of protons from a hydrolysis reaction of by-products of the polymerisation reaction with one or more enzymes; monitoring an electrical output signal derived from an ISFET exposed to the reaction chamber; and correlating changes in an output signal with said reactions between the unknown polynucleic acid and said known reagents to thereby identify the unknown nucleic acid.

### FIELD OF THE INVENTION

The present invention relates to a method for increasing the detectable ionic signal from a chemical reaction, particularly though not exclusively methods for DNA sequencing and SNP identification.

### BACKGROUND OF THE INVENTION

Sequencing of nucleic acids can be accomplished by synthesising all or part of a nascent nucleic acid strand, in a polymerisation reaction between the unknown (target) polynucleic acid and the free nucleotides dATP, dCTP, DGTP, DTTP added one at a time. Identification is traditionally done by then monitoring the main product of such polymerisation reaction, namely the newly synthesised polynucleic acid by gel electrophoresis, and direct or indirect optical quantification.

Subsequently, work in the field of sequencing and identifying of nucleic acids has examined the ability of an ION Sensitive Field Effect Transistor (ISFET) to detect nucleotide incorporation to a nucleic acid strand by detecting the change in pH resulting from the reaction. Typically, Hydrogen ions (protons) are released during the reaction. The electrical output signal strength of the ISFET depends on the amount of hydrogen ions released, which largely depends on the quantity of nucleic acid (for instance RNA or DNA) present in the sample to be measured. In some cases, the quantity of protons released directly from the incorporation reaction may be too small to be detected accurately by the ISFET and signal processing. Additionally, there may be high levels of background signal, which may be a result of by-products of the reaction. The background signal created by said by products is therefore been considered a nuisance.

In the following discussion, reference to protons, hydrogen ions, and H⁺are intended to be synonymous, all associated with the pH of a fluid.

In 1992 Toshinari Sakurai (“Real-Time Monitoring of DNA Polymerase Reactions by a Micro ISFET pH sensor”, 1992, 64, 1996-1997, Analytical Chemistry) described how an ISFET could be used to monitor the kinetics of a DNA polymerase reaction with a ISFET. He postulated that the incorporation of a dNTP onto a strand of DNA consumes a hydrogen ion to produce a growing DNA strand and pyrophosphate. The change in pH was measured by the ISFET.

Nine years later DNA Electronics developed a method whereby DNA could be identified by detecting the change in pH during the incorporation of nucleotides onto a growing nucleic acid strand. The observations suggested that hydrogen ions were released. Victorova et el (“New Substrates of DNA polymerases”, Federation of European Biochemical Societies Letters, 453,pp 6-10, 1999) discussed how, in vivo, pyrophosphatase (PPase) breaks down pyrophosphate. However, bodily fluids are too heavily buffered to observe any pH change from such a reaction. Citing Victorova in a corresponding patent publication (WO03/073088), it was suggested that pyrophosphatase bound to polymerase would hydrolyse PPi to produce orthophosphate and hydrogen ions.

Later EP2304420 (Ion Torrent) disclosed a device having an array of chemFETs comprising a passivation layer attached to pyrophosphate receptors to detect the PPi.

These efforts have concentrated on detecting protons released directly from the incorporation reaction or detecting by-products of said reaction. However, to-date, no one has considered adding enzymes to break down the by-products to release and detect more protons in addition to those produced during the incorporation of nucleotides.

It is an object of the present invention to provide a method which increases the concentration of target analytes for detecting a chemical reaction by deconstructing by-products of a DNA reaction. The chemical reaction is preferably the addition or removal of a particular nucleic acid/nucleotide/nucleoside (the terms being used interchangeably herein).

### SUMMARY OF THE INVENTION

According to a first aspect of the invention there is provided a method of identifying an unknown nucleic acid comprising:
combining the unknown polynucleic acid with known nucleic acid reagents in a reaction chamber;
producing a first quantity of protons from a polymerisation reaction if the bases of one or more of the unknown nucleic acids are complementary to the bases of one or more known nucleic acids comprised within the known reagents;
producing a second quantity of protons from a hydrolysis reaction of by-products of the polymerisation reaction with one or more enzymes;
monitoring an electrical output signal derived from an ISFET exposed to the reaction chamber; and
correlating changes in an output signal with said reactions between the unknown polynucleic acid and said known reagents to thereby identify the unknown nucleic acid.

Preferably, the method comprises determining if there is a significant change in the output signal and correlating only such significant changes. Preferably, the known nucleotide is one of dATP, dGTP, dTTP, or dCTP and the known nucleic acid is a primer.

A method according to any preceding claim , wherein the steps of the method are repeated to sequence the unknown nucleic acid, each repeat using one of dATP, dGTP, dTTP, or dCTP as the known nucleotide.

It is also preferred that the known nucleic acid is an allele-specific primer. Preferably, the polymerisation reaction is an allele-specific amplification reaction

Preferably, one of the by-products is pyrophosphate and the one or more enzymes comprise a pyrophosphatase. The pyrophosphate may preferably be added to the reaction chamber before the polymerisation reaction starts, such that the polymerisation reaction and deconstruction reaction are substantially concurrent.

Alternatively, it is preferred that one of the by-product is DNA and one or more of the enzymes comprises an enzyme to deconstruct DNA. Preferably, the one or more enzymes comprise one or more of an exonuclease, preferably selected from the group consisting of: T7 Exonuclease, RecJf, Exonuclease I, Exonuclease T, and BAL-31 Exonuclease, Exonucelase III or Lambda Exonuclease.

The one or more enzymes may preferably be added to the reaction chamber after the polymerisation reaction starts such that the deconstruction reaction occurs after the polymerisation reaction.

The electrical output signal may preferably be measured differentially between the ISFET and another ISFET or MOSFET.

Determining if there is a significant change in the output signal may preferably comprise determining a magnitude of an electrical output signal change, or more preferably comparing the change in the electrical output signal to a threshold signal change value. However, determining if there is a significant change in the output signal may preferably comprise evaluating the change in the output signal from the polymerisation reaction separately from the change in the output signal from the deconstruction reaction.

Determining if there is a significant change in the output signal may also comprise comparing the output signal change to an output signal change where the polymerisation reaction does not occur because the one or more nucleotides of the unknown nucleic acid are not complementary to the known nucleotide and/or known nucleic acid.

An advantage of the present invention is therefore an increase in the signal strength by detecting protons from both the polymerisation and deconstruction reactions.

It is particularly preferred that the enzyme hydrolyse pyrophosphate, but not triphosphate acid. Thus, in their presence, a greater pH change can be detected (on top of that from the hydrolysis of dNTP).

An allele-specific primer for an SNP may be extended by addition of Nucleoside TriPhosphates (dNTPs or ddNTPs) during DNA polymerisation. The extended/polymerised strand is optionally amplified and then an exonuclease can be added to remove the newly-added nucleotides (or their amplified equivalents) one by one. In the simplest case, it is preferred that only one NTP is added and then only one is later removed. The H⁺ions released on both NTP addition and subsequent removal are detected by the ISFET(s).

Exonuclease activity on non-target DNA (for instance the original primer) can preferably be ameliorated by capping or modification. Where there is amplification of the target strand, exonuclease activity on non-target DNA is largely irrelevant as it is swamped by the amplified target—this applies to the detection of SNPs in particular, so amplification is particularly preferred for this aspect. In the case of simple sequencing to determine the identity of a portion of a target strand, amplification is probably not always necessary, but if exonuclease is to be used in such sequencing, then the primer that is extended to initiate the sequencing may preferably be capped or modified to prevent exonuclease activity against it.

The present invention thus provides a method of identifying a nucleic acid. This involves determining its identity, for instance that of its base: i.e. is it C, G, T A or U. This may preferably be part f a method of sequencing a strand of DNA or other genetic material. It may be preferable to use only a PPase to assess levels of PPi produced on addition by polymerisation of a nucleoside to a nascent strand: this may be repeated many times to sequence a long stretch or occur only once. In the latter ascpet, the invention thus also applies to a method of determining the identity of a single nucleotide, in either a larger sequencing effort or in the detection of an SNP. When looking at just a single nucleotide addition, it may also be useful to include an exonuclease activity to remove the added nucleoside to further increase accuracy, although this can be applied to longer sequences as well.

It is particularly preferred that when sequencing a template or target strand, this is done in the presence of only one type of nucleoside triphosphate, say Cytosine (C). The addition of that nucleoside will trigger H+ ion release for detection by the ISFET and indicate that corresponding position on the template strand is the complementary nucleic acid, in this instance G.

The same holds for SNP detection. However, in that case, or where it is desired to detect a the identity of broader stretch of nucleic acids in the template, then it is also particularly preferred expose the template or target strand to primers. In the case of SNP detection, these are allele-specific primers. Exposure or contact with primers in the presence of suitable nucleoside triphosphates (i.e. all or at least A, T (or U), C and G) and polymerase will lead to annealing (“recognition”) of the primer to the target strand at the SNP (or other desired) site followed by chain elongation due to polymerisation. Such a “match” will lead to hydrogen ion release, detectable by the ISFET. The exonuclease activity is most preferably used in respect of SNP's, particularly to confirm the addition of a nucleoside at the end of a primer, where said end is designed to overly exactly the position of the SNP such that the first or next nucleoside added to the 3′ end of the primer will be that complementary to the SNP. Capping and modification of the primer are also helpful here to prevent the primer being acted on.

### BRIEF DESCRIPTION OF THE DRAWINGS

Specific embodiments of the invention will now be described by way of example only with reference to the accompanying figures, in which:

FIG. 1 is an exemplary flow chart of a method according to an embodiment of the invention employing a pyrophosphatase (PPase) enzyme;

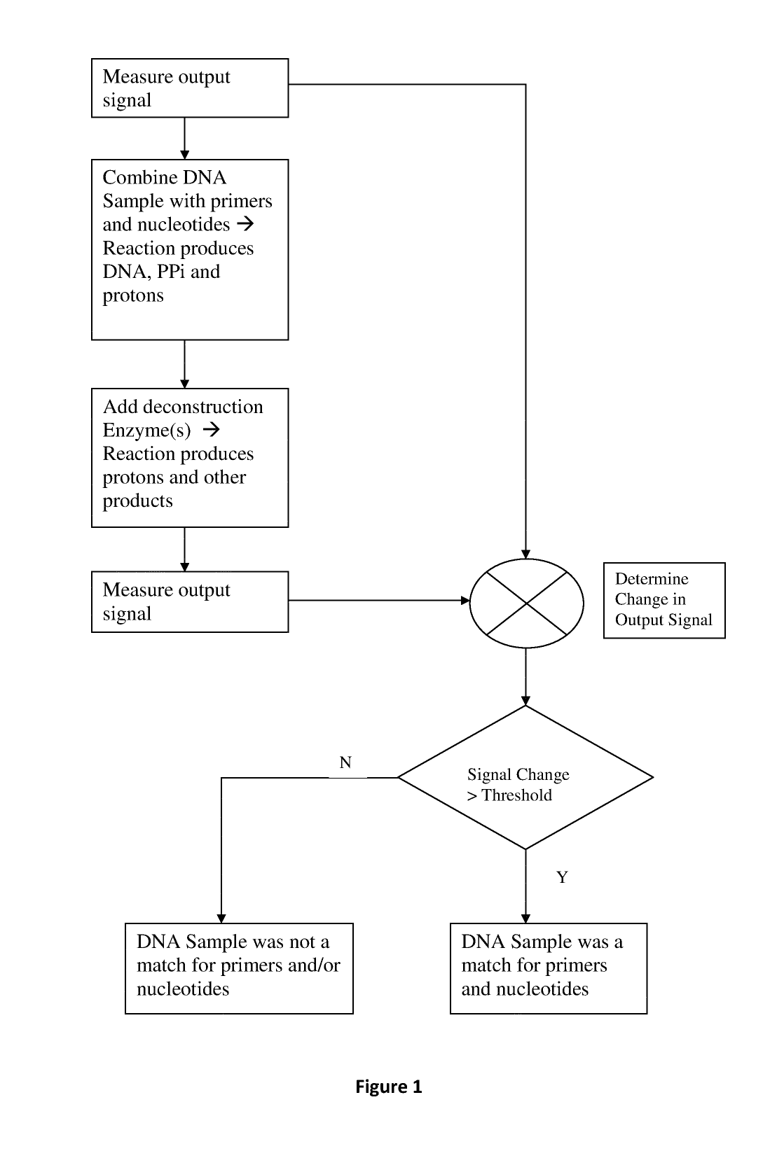

FIG. 2 is a graph of change in pH over time for various reactions in both the presence and absence of pyrophosphate (PPi) and pyrophosphatase (PPase);

FIG. 3 is a graph of change in pH over time for various reactions according to an embodiment of the invention employing an exonuclease, in this instance Lambda Exonuclease; and

FIG. 4 is a graph of change in pH over time for various reactions according to an embodiment of the invention employing an exonuclease, in this instance Exonuclease III.

### DETAILED DESCRIPTION

Described herein is a detection method which monitors deconstruction reactions associated with a newly-synthesised nucleic acid. The substrate of this deconstruction reaction arises from nucleic acid polymerisation (the primary reaction) which itself release hydrogen ions as nucleosides are added to (i.e. polymerised to form) an extending chain. Thus, the deconstruction reaction is referred to as the ‘secondary reaction’. This detection of further hydrogen ions released from the secondary reaction can be applied alone, or in addition to the detection of the hydrogen ions released from the primary reaction. The creation of a secondary reaction, which involves catalysis (principally hydrolysis) of the by-product of primary reaction, initiates an additional secondary signal to the primary signal. Any secondary reaction that is detected by the same detector as the primary reaction can be viewed as a signal enhancement in situ. Hydrogen ions released from secondary reaction can also be detected by a separate detector.

In other words, the aim is signal enhancement, thus enabling increased accuracy over the background. This is achieved by in fact harnessing the background.

Hydrogen ions released from the secondary reaction are preferably detected by the same (i.e. a single) ISFET sensor as the primary reaction, but it could also be a further sensor, for example if two sensors per reaction chamber are used or if the primary and secondary reactions occur at different times or locations.

A deconstruction reaction is for instance a reaction, preferably catalysed by the present enzymes, that produces one or more break-down products from a substrate. For instance, the preferred enzymes are pyrophosphates or exonucleases, both of which are hydrolases which catalyze the hydrolysis of phosphate bonds, thus deconstructing the phosphate bond to ultimate yield more Hydrogen ions. Thus, it is particularly preferred that the deconstruction enzymes are those capable of hydrolysing phosphate/phosphodiester bonds, especially in the context of nucleic acids (i.e. nucleic acid phosphodiester bonds).

Preferably, the (primary) chemical reaction is the synthesis of a nascent polynucleotide strand (being two or more nucleotides) of which RNA and DNA are particularly preferred. It is preferred that the fluctuations of ionic charge indicate the insertion of di-deoxynucleotide triphosphates (ddNTP) and deoxynucleotide triphosphates (dNTP) onto the nascent polynucleotide strand. The nascent polynucleotide strand may be an entirely new polynucleotide strand or, alternatively, the newly added sections of an annealed strand such as a primer, added during the preferred sequencing reaction.

Preferably, the nascent polynucleotide strand is the complement of a template strand.

The invention is described herein with reference to DNA, but applies equally to other nucleic acids.

DNA sequencing using an embodiment of the invention is performed as follows, with reference to DNA as the preferred polynucleotide. A quantity of DNA of interest is amplified using either a polymerase chain reaction or cloning or other amplification technique, and the region of interest is primed, e.g. using an mRNA primer. DNA polymerase catalyses DNA synthesis (polymerisation) through the incorporation of nucleotide bases in a growing DNA chain (for instance the nascent strand referred to herein), such as primer extension.

ISFETs are known in the art. Preferably, the ISFET used herein is provided with an ion sensitive layer such as silicon nitride, on top of which a layer of polymerase may be provided. The magnitude of pH change is detected in order to reliably detect nucleotide insertion (elongation of the nascent polynucleotide strand). The change in electrical signal of the ISFET is indicative of nucleotide insertion during DNA synthesis. Upon insertion of a nucleoside triphosphate to a nascent strand, a pyrophosphate (PPi or P₂O₇⁴⁺) ion is released. Nucleic acid polymerisation can be found in many biological reactions such as cell replication, gene expression, or DNA repair. The reaction generally occurs with the help of enzymes (e.g. polymerase). Technologies have been developed to replicate the nucleic acid replication reaction outside host cells or organisms. This has many applications such as amplification of nucleic acid in vitro, sequencing of nucleic acid, genotyping, and molecular diagnosis. A typical nucleic acid polymerisation can involve triphosphate deoxynucleotides and a DNA substrate as expressed below.

xdNTP+DNAy→xPPi+DNA(x+y)+H⁺

x molecules of dNTPs are hydrolysed into x molecules of pyrophosphate and the DNA is extended from y nucleotides to x+y nucleotides. The reaction can also involve nucleotides and RNA and the nucleic acid substrate can be double stranded or single stranded. The actual number of hydrogen ions released depending on various factors such as pK values of the reagents.

This polymerisation reaction occurs when the 5′ end of a nucleotide is incorporated onto the 3′ (extending) end of a polynucleic acid annealed to a second (template or target) polynucleic acid, the sequence or nucleic acid identity of which is to be determined. The incorporation will only occur if the nucleotide is complementary to the base of the second polynucleic acid (i.e. the template strand) opposite the point of incorporation (i.e. Thymine/Uracil is complementary to Adenine and Guanine is complementary to Cytosine).

In this reaction, the newly synthesized (e.g. DNA) nascent strand and PPi are considered by-products, the main product of interest being hydrogen ions. However, we have surprisingly discovered that these oft-neglected by-products may be deconstructed to produce further hydrogen ions to thereby enhance the accuracy of the sensed reaction. The complete chemical system is thus:

dNTP+DNA(y)→PPi+DNA(y+1)+H⁺ (primary change in pH)

PPi+PPase→2Pi+PPase+H⁺ (secondary change in pH)

DNA(y+1)+Exonuclease→dMTP+Exonuclease+H⁺ (secondary change in pH according to a further embodiment that can be used in addition to or separately from the deconstruction of PPi)

The primary reaction is dependent on the DNA successfully polymerising, which occurs if the nucleotides or primer added are complementary to the unknown template strand of the DNA to be sequenced (i.e. the bases of one or each of nucleotide are identified, for example as being C, G, A, T, or U). The secondary reactions depend on by-products being produced by the primary reaction. Therefore, the overall reaction produces (i.e. results in a net release of) protons if the nucleotides or primer added are complementary to the unknown template DNA. These protons are detected by the ISFET and correlated to the addition of the specific nucleotides and primer to identify the original DNA in the sample. Thus, knowing which nucleotide or primer was added to extend the nascent strand will indentify a portion of the unknown (template) nucleic acid.

The polymerisation reaction may result from hybridising the unknown nucleic acid with an allele specific primer and mixed dNTPs. Alternatively, a primer may be used to anneal to the unknown nucleic acid up to a location of interest followed by adding a known dNTP. In a further alternative, the polymerisation reaction may be an allele-specific amplification reaction using PCR or isothermal amplification. The use of an allele-specific primer allows for identifications such as SNPs, i.e. the determination of the presence or absence of a particular SNP of the identity of the nucleotide at the relevant position, to thereby identity an allele in a sample.

The hydrolysis of dNTP to PPi causes changes in proton concentration within certain pH ranges and with the presence of divalent metal ions, such as magnesium or manganese.

The hydrolysis of pyrophosphate produces two phosphates. The hydrolysis of pyrophosphate at certain pH ranges and in the presence of divalent metal ions, such as Magnesium or Manganese, results in a similar pH change to dNTP hydrolysis.

Inorganic PPase may be used to catalyze the hydrolysis of inorganic PPi to form orthophosphate, as expressed below:

P₂O₇⁻⁴+H₂0^{(PPiase})→2HP0₄⁻²+H⁺

Pyrophosphate is generally stable for a day or longer without the help of enzymes. Preferred reactions are polymerase chain reaction, sequencing, or primer extension. In such reactions, the pyrophosphate remains stable in the reaction mixture. pH change is a function of the reaction and reaction percentage. By adding an enzyme or enzymes that hydrolyse pyrophosphate, but not triphosphate acid, a greater pH change can be added (on top of that from the hydrolysis of dNTP).

It is important that the enzyme that catalyses the hydrolysis of pyrophosphate does not attack triphosphate. In many technologies, the hydrolysis of triphosphate is strictly linked with the presence of target molecules or specific reactions.

Pyrophosphatases are acid anhydride hydrolases that act upon diphosphate bonds. Enzymes such as inorganic pyrophosphatase or thermostable inorganic pyrophosphatase fulfill such criteria. Pyrophosphatases are widely available and may be mixed with polymerase in the polymerisation reaction. In the case of sequencing, the pyrophosphatase is used in tandem with a DNA polymerase to increase the pH change.

As PPi was previously thought to be merely a by-product of DNA polymerisation, best removed or ignored to avoid complications with the detection, and capable of inducing background noise, we were the first to identify that its inclusion can have beneficial effects in terms of detection accuracy if harnessed.

In one embodiment, a thermostable pyrophosphatase is mixed with thermostable DNA polymerase in a PCR reaction. The pyrophosphatase catalyses the hydrolysis of PPi, which is the product of polymerisation. The hydrolysis of the pyrophosphate, previously considered a waste product at best, thus increases the total pH change (i.e. the concentration of hydrogen ions) from a single nucleotide addition. Thus, a method of PCR amplification comprising use of PPase to augment or enhance signal detection from an ISFET upon nucleoside addition is also provided.

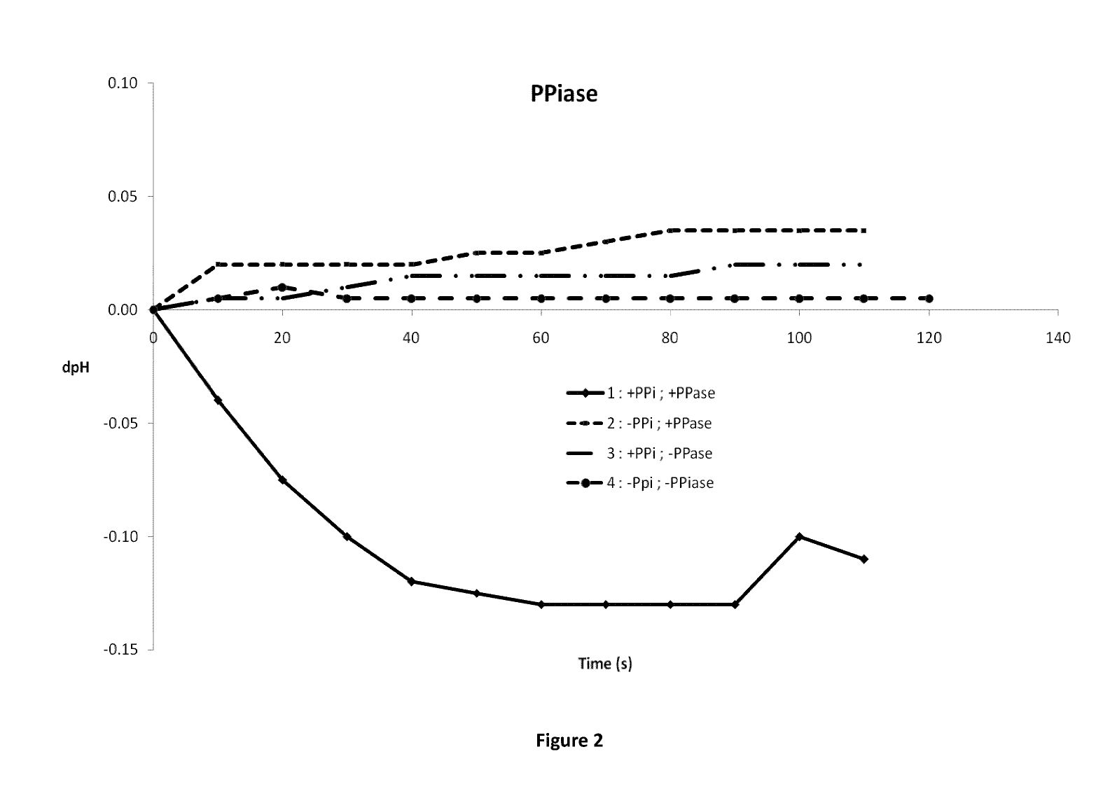

The effect of the reaction is demonstrated in the FIG. 2. The change in pH is measured for 350 seconds for 4 reactions. Line 4 shows a reaction having neither

Pyrophosphate nor pyrophosphatase; the pH changes very little. Line 2 shows a reaction having no Pyrophosphate but some pyrophosphatase; the pH changes initially in reaction to the added pyrophosphatase buffer but then decreases until equilibrium, the offset merely indicative of the buffer addition and not proton release from a reaction. Line 1 shows a reaction having both Pyrophosphate and pyrophosphatase; the change in pH increases until equilibrium.

The reagents used in the reaction comprise KCl, MgCl2, PPiase. Preferred concentrations are provided below.

Preferably, the concentration of KCl is greater than 10 mM, more preferably greater than 40 mM, 50 mM, 80 mM, 100 mM or 120 mM. Preferably said concentration is less than 500 mM, more preferably less than 400 mM, 300 mM, or 200 mM. Any combination of these upper and lower limits of these is envisaged.

Preferably, the concentration of MgCl2 is greater than 1 mM, more preferably greater than 2 mM, 3 mM, or 4 mM. Preferably said concentration is less than 10 mM, more preferably less than 8 mM, 7 mM, or 5 mM. Any combination of these upper and lower limits is envisaged.

Preferably, the amount of PPiase in a 50 uL reaction volume is greater than 0.01 U (where U is defined by an unit of enzymatic activity under conditions prescribed by the manufacturer), more preferably greater than .05 U, 0.1 U, 1 U or 10 U. Suggested pyrophosphatase compounds include Inorganic PPiase made from E. coli or yeast and/or Thermostable Inorganic PPiase both available from New England Biolab (Catalogue no.M0361S is preferable). Excessive commercially available PPiase could lead to buffering which is undesirable in the detection of hydrogen ions. Ideally PPiase is provided without any buffer components.

A typical reaction may occur in a micro-chamber having a volume from 1 nL to 100 uL, but the volume may be bigger depending on the size of the ISFET and sample volume available.

The temperature of the reaction volume may be 18-45° C., preferably greater than 25° C., 30° C., or 35° C., preferably less than 40° C. or 38° C. Any combination of these upper and lower limits is envisaged.

The pH of the fluid including the DNA sample and reagents after nucleotide incorporation is preferably between 7 and 8.6; more preferably above pH 7.5 or above pH7.9; preferably below pH 8.4 or pH 8.1. Any combination of these upper and lower limits is envisaged. NaOH may be added to the fluid as required to achieve the above pH setting. Other appropriate buffers are also contemplated.

In addition to, or separate to, the deconstruction of PPi, the by-product DNA may also be deconstructed to release protons. The reaction may be expressed as:

DNA→dNMP+H⁺

where dNMP is a deoxymonophosphate and the DNA is a newly synthesised (nascent) polynucleic acid complementary to the unknown DNA template.

Thus. the present enzyme may be a DNA deconstruction enzyme preferably a hydrolysis enzyme such as an exonuclease, preferably being capable of using DNA and/or RNA as its substrate. Any reference to DNA herein also applies to RNA and other polynucleotides unless otherwise apparent.

Because the DNA deconstruction enzyme (i.e. an exonuclease) typically breaks down any DNA and the purpose is to identify the DNA or portion thereof, the DNA deconstruction enzyme enzyme is added after the production of identifiable DNA. In this way, protons released from the deconstruction reaction represent those derived from deconstruction (preferably hydrolysis) of the identifiable DNA and not other DNA fragments in the reaction mixture. DNA is identifiable when produced from an allele-specific polymerisation reaction. If the unknown DNA does not polymerise there will still be some deconstruction activity so it is preferable to amplify the quantity of identifiable DNA. This may be done using allele specific amplification techniques such that the quantity of DNA that can be identified is orders of magnitude greater than the unamplified DNA.

The reagents used in the reaction comprise KCl, MgCl2, Bovine serum albumin (BSA), and DNA deconstruction enzyme. Preferred concentrations are provided below.

Preferably the concentration of KCl is greater than 10 mM, more preferably greater than 10 mM, 20 mM, 30 mM, 40 mM or 50 mM. Preferably said concentration is less than 500 mM, more preferably less than 400 mM, 300 mM, or 200 mM. Any combination of these upper and lower limits is envisaged.

Preferably the concentration of MgCl2 is greater than 1 mM, more preferably greater than 2 mM, 3 mM, or 4 mM. Preferably said concentration is less than 10 mM, more preferably less than 8 mM, 7 mM, or 5 mM. Any combination of these upper and lower limits is envisaged.

A typical reaction may occur in a micro-chamber having a volume from 1 nL to 100 uL, but the volume may be bigger depending on the size of the ISFET and sample volume available.

The temperature of the reaction volume may be 18-50° C., preferably greater than 25° C., 30° C., or 35° C., preferably less than 40° C. or 38° C. Any combination of these upper and lower limits is envisaged.

The pH of the fluid including the DNA sample and reagents after nucleotide incorporation is preferably between 7 and 9; more preferably above pH 7.5, pH 8, or pH 8.3; preferably below pH 9, pH 8.6 or pH 8.5.NaOH may be added to the fluid as required to achieve the above pH setting. Any combination of these upper and lower limits is envisaged.

The concentration of BSA is between 0.1 and 10 mg/ml, preferably greater than 0.2 mg/ml, 0.5 mg/ml, or 1.0 mg/ml; preferably less than 5 mg/ml, 2.0 mg/ml or 1.5 mg/ml. Any combination of these upper and lower limits is envisaged.

According to the embodiment of the DNA deconstruction enzyme, the enzyme used may be any enzyme that deconstructs DNA, preferably by the hydrolysis of phosphate/phosphodiester bonds, and releases protons. Examples of such enzymes are exonucleases. Exonucleases are enzymes that work by cleaving nucleotides one at a time from the end (exo) of a polynucleotide chain via a hydrolyzing reaction that cleaves phosphodiester bonds at either the 3′ or the 5′ end. T7 Exonuclease, RecJf, Exonuclease I, Exonuclease T, and BAL-31 Exonuclease. Preferably, the enzyme is Exonucelase III or Lambda exonuclease, both available from New England Biolab or Fermentas. Preferred examples are Exonuclease III from Fermentas (EN0191) and Lambda Exonuclease from Fermentas (EN0562).

Preferably, the quantity of Exonuclease per 50 ul is greater than 10 U, 20 U, 50 U, or 100 U. Preferably the quantity of Lambda Exonuclease per 50 ul is greater than 1 U, 2 U, 5 U, or 10 U.

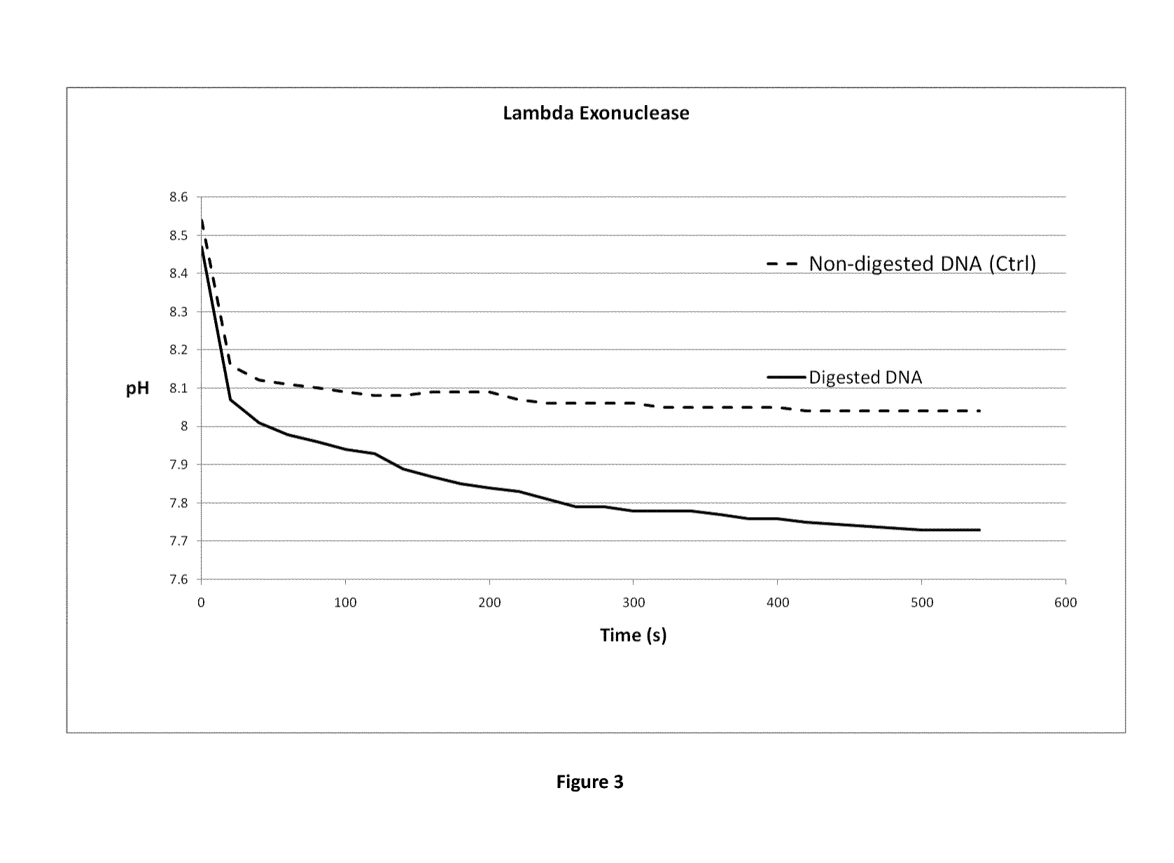

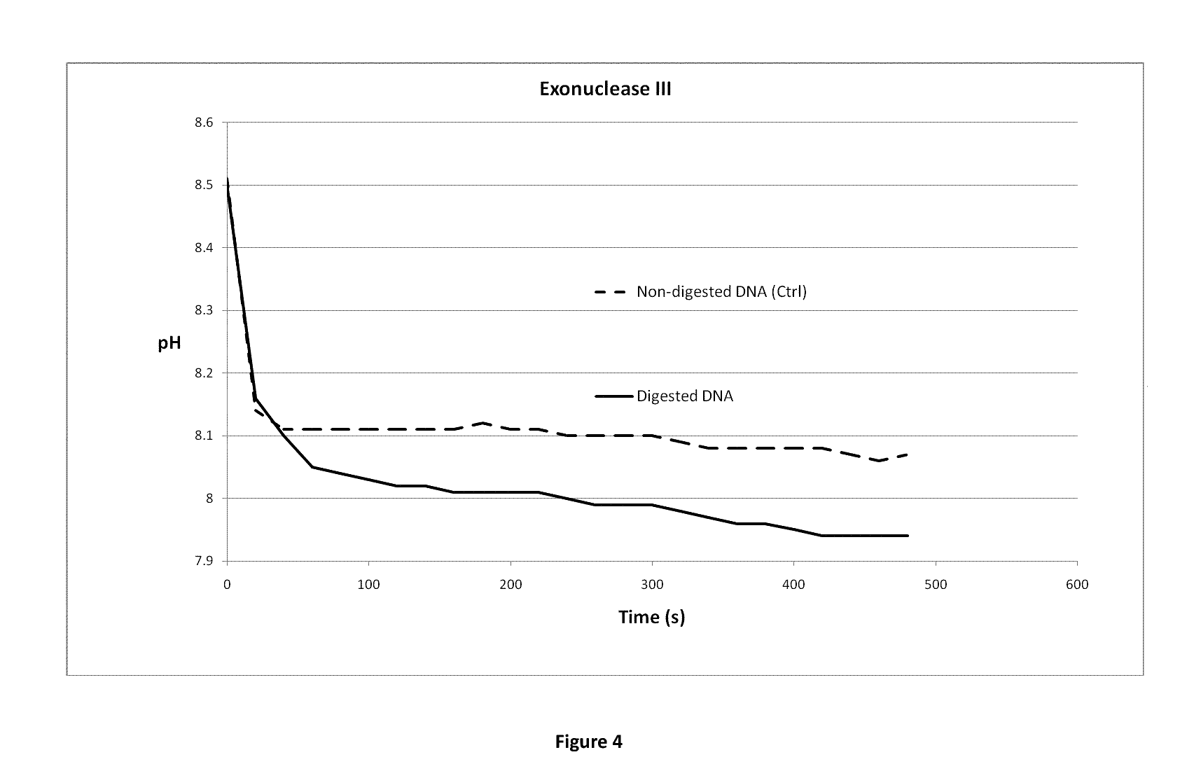

FIG. 4 is a graph of the pH of a reaction chamber with and without Exonuclease III. In the reaction 10 ng/uL of dsDNA substrate was mixed with Exonuclease III to produce protons with the shown effect on pH (shown by the digested DNA—solid line). The control experiment (shown by the non-digested DNA—dotted line). contains all reagents except the enzyme. As shown, the pH will initially drop due to the addition of reagents to the reaction chamber thereafter becoming relatively unchanged for the control or dropping further for the enzyme reaction. FIG. 3 shows that lambda Exonuclease has a similar effect.

The amount of DNA present in the sample to be tested may vary or be unknown but is typically between 50 and150 ng/uL preferably more than 10 ng/uL, more than 20 ng/uL or more than 50 ng/uL.

Nucleic acid deconstruction may be used to enhance the pH signal from samples comprising purified nucleic acid, or nucleic acid from a previous enzymic reactions. Therefore the method can tolerate background compounds, such as leftover primers/probes (up to 1 microM), oligo nucleotide (up to 10 mM), DNA polymerases, and other reaction components or by-products.

Although the invention has been described in terms of preferred embodiments as set forth above, it should be understood that these embodiments are illustrative only and that the claims are not limited to those embodiments. Those skilled in the art will be able to make modifications and alternatives in view of the disclosure which are contemplated as falling within the scope of the appended claims. Each feature disclosed or illustrated in the present specification may be incorporated in the invention, whether alone or in any appropriate combination with any other feature disclosed or illustrated herein. For example, the enzymes to deconstruct by-products may comprise both pyrophosphatase and an enzyme to deconstruct DNA, to release protons from the primary polymerisation reaction and both deconstruction reactions, such that the signal change is even larger.

The terms nucleotide and nucleic acid (whether poly- or single) are used interchangeably herein. Either is preferred.

The present invention, therefore provides a means of enhancing an ionic signal, i.e. a change in pH caused by release of hydrogen ions, such release being indicative of an addition or the removal of a nuclei acid from a nucleic acid strand. By enhancing the signal due to increased hydrogen ion release associated with PPase or exonuclease activity for instance), the accuracy of the determination of the identity of that particular (unknown) nucleic acid can significantly improved. It is already known in the art how the signal from an ISEFT is correlated to the identity of that particular (unknown) nucleic acid.

1-16. (canceled)

17. A method of identifying an unknown nucleic acid comprising the steps of:
providing a reaction mixture comprising a pyrophosphatase, a polymerase, the unknown nucleic acid and known nucleic acid reagents to a reaction chamber, wherein the pH of the reaction mixture is above 7.5;
producing a first quantity of protons from a polymerization reaction if the bases of one or more of the unknown nucleic acids are complementary to the bases of one or more known nucleic acids comprised within the known reagents;
producing a second quantity of protons from a hydrolysis reaction of pyrophosphate with the pyrophosphatase, wherein the pyrophosphate is a by-product of the polymerization reaction;
monitoring an electrical output signal derived from total protons produced from both the first and the second reactions using an ISFET exposed to the reaction chamber; and
correlating changes in an output signal with said reactions between the unknown nucleic acid and said known reagents to thereby identify the unknown nucleic acid.

18. The method according to claim 17 further comprising determining if there is a significant change in the output signal and correlating only such significant changes.

19. The method according to claim 17, wherein the known nucleotide is one of dATP, dGTP, dTTP, or dCTP and the known nucleic acid is a primer.

20. The method according to claim 17, wherein the steps of the method are repeated to sequence the unknown nucleic acid, each repeat using one of dATP, dGTP, dTTP, or dCTP as the known nucleotide.

21. The method according to claim 17, wherein the known nucleic acid is an allele-specific primer.

22. The method according to claim 17, wherein the polymerization reaction is an allele-specific amplification reaction.

23. The method according to claim 17, wherein the pyrophosphatase is added to the reaction chamber before the polymerization reaction starts, such that the polymerization reaction and pyrophosphate hydrolysis reaction are substantially concurrent.

24. The method according to claims 17, wherein the pyrophosphatase is added to the reaction chamber after the polymerization reaction starts such that the pyrophosphate hydrolysis reaction occurs after the polymerization reaction.

25. The method according to claim 17, wherein the electrical output signal is measured differentially between the ISFET and another ISFET or MOSFET.

26. The method according to claim 18, wherein determining if there is a significant change in the output signal comprises determining a magnitude of an electrical output signal change.

27. The method according to claims 18, wherein determining if there is a significant change in the output signal comprises comparing the change in the electrical output signal to a threshold signal change value.

28. The method according to claims 18, wherein determining if there is a significant change in the output signal comprises comparing the output signal change to an output signal change where the polymerization reaction does not occur because the one or more nucleotides of the unknown nucleic acid are not complementary to the known nucleotide and/or known nucleic acid.

29. A method of identifying an unknown nucleic acid comprising the steps of:
combining the unknown nucleic acid with known nucleic acid reagents in a reaction chamber, wherein the pH of the reaction mixture is above 7.5;
producing a first quantity of protons from a polymerization reaction if the bases of one or more of the unknown nucleic acids are complementary to the bases of one or more known nucleic acids comprised within the known reagents;
producing a second quantity of protons from a hydrolysis reaction of by-products of the polymerization reaction with one or more enzymes;
monitoring an electrical output signal derived from total protons produced from both the first and the second reactions using an ISFET exposed to the reaction chamber; and
correlating changes in an output signal with said reactions between the unknown nucleic acid and said known reagents to thereby identify the unknown nucleic acid.

30. The method according to claim 29 further comprising determining if there is a significant change in the output signal and correlating only such significant changes.

31. The method according to claim 29, wherein the known nucleotide is one of dATP, dGTP, dTTP, or dCTP and the known nucleic acid is a primer.

32. The method according to claims 29, wherein the steps of the method are repeated to sequence the unknown nucleic acid, each repeat using one of dATP, dGTP, dTTP, or dCTP as the known nucleotide.

33. The method according to claim 29, wherein the known nucleic acid is an allele-specific primer.

34. The method according to claim 29, wherein the polymerization reaction is an allele-specific amplification reaction.

35. The method according to claim 29, wherein one of the by-products is pyrophosphate and the one or more enzymes comprise a pyrophosphatase.

36. The method according to claim 35, wherein the pyrophosphatase is added to the reaction chamber before the polymerization reaction starts, such that the polymerization reaction and pyrophosphate hydrolysis reaction are substantially concurrent.

37. The method according to claim 29, wherein one of the by-products is DNA and one or more of the enzymes comprises an enzyme to deconstruct DNA.

38. The method according to claim 37, wherein the one or more enzymes comprise one or more of: an exonuclease, preferably selected from the group consisting of: T7 Exonuclease, RecJf, Exonuclease I, Exonuclease T, and BAL-31 Exonuclease, Exonuclease III or Lambda Exonuclease.

39. The method according to claim 29, wherein the one or more enzymes is added to the reaction chamber after the polymerization reaction starts such that the deconstruction reaction occurs after the polymerization reaction.

40. The method according to claim 29, wherein the electrical output signal is measured differentially between the ISFET and another ISFET or MOSFET.

41. The method according to claim 30, wherein determining if there is a significant change in the output signal comprises determining a magnitude of an electrical output signal change.

42. The method according to claim 30, wherein determining if there is a significant change in the output signal comprises comparing the change in the electrical output signal to a threshold signal change value.

43. The method according to claim 30, wherein determining if there is a significant change in the output signal comprises comparing the output signal change to an output signal change where the polymerization reaction does not occur because the one or more nucleotides of the unknown nucleic acid are not complementary to the known nucleotide and/or known nucleic acid.
